(19) United States
(12) Patent Application Publication
(10) Pub. No.: US 2025/0091749 A1
(43) Pub. Date: Mar. 20, 2025

(54) CONTAINER TREATMENT SYSTEM WITH IMPROVED PERSONAL PROTECTION

(71) Applicant: KRONES AG, Neutraubling (DE)

(72) Inventors: Tobias EHRISMANN, Burglengenfeld (DE); Stefan SCHERL, Bernhardswald (DE)

(21) Appl. No.: 18/888,410

(22) Filed: Sep. 18, 2024

(30) Foreign Application Priority Data
Sep. 18, 2023 (DE) 20 2023 105 407.2

(51) Int. Cl. B65B 57/00

(52) U.S. Cl. CPC B65B 57/005

(57) ABSTRACT

A container treatment system comprising a container treatment device with a treatment region for treating containers and a protective enclosure which at least partially encloses the treatment region. The container treatment system further comprises a sensor device which is configured to detect an intrusion into a predefined protective region in an environment of the container treatment device. The container treatment system further comprises a processing device that is configured to adapt an operation of the container treatment device and/or the container treatment system depending on a signal output of the sensor device.

### CROSS REFERENCE TO RELATED APPLICATIONS

This application claims the benefit under 35 U.S.C. § 119 (a) of German Patent Application No. DE 20 2023 105 407.2, filed Sep. 18, 2023 entitled CONTAINER TREATMENT SYSTEM WITH IMPROVED PERSONAL PROTECTION, and whose entire disclosure is incorporated by reference herein.

### TECHNICAL FIELD

The invention relates to a container treatment system with a container treatment device which has a protective enclosure.

### TECHNICAL BACKGROUND

In a container treatment system, a container treatment device can be equipped with a physical machine protector (protective enclosure). This machine protector is intended to prevent people from being injured, e.g. by crushing or burning, by moving parts directly at the container treatment device during production. The machine protector can also prevent parts or media from escaping in an undesired manner from the container treatment device, which could also injure people.

DE 20 2018 101 090 U1 discloses a labeling machine comprising a carousel and a labeling unit that can be arranged exchangeably on the periphery of the carousel. The labeling unit comprises a fixed base, a functional part that can be moved relative to the base in the direction of the carousel, and a unit protector that protects the label transfer region from user intervention when the labeling unit is in a coupled state. The unit protector comprises at least one protective element attached in stationary fashion to the base and a protective element that can be moved relative to this protective element in the direction of the carousel.

DE 10 2017 215 467 A1 discloses a container treatment system comprising a machine region which is at least partially enclosed by a protective covering, and a unit which cooperates with the machine region through an opening in the protective covering.

A disadvantage of conventional protection technology can be that the machine protector often cannot remain active outside of operating hours, e.g. during a refitting or exchanging process, because external intervention is necessary, or certain materials have to “pass through” the machine protector. Therefore, for example, a door or a protective plate of the machine protector must be opened. This poses a risk of injury, especially in semi- or fully automatic exchange mode where no person actually needs to be present since it cannot be ruled out that a person might reach in. Even with very slow movements of the container treatment device, a residual risk of injury cannot be excluded.

The invention is based upon the object of creating an improved technique for protecting persons from injury by a container treatment device.

### SUMMARY OF THE INVENTION

The object is achieved by the features of independent claim 1. Advantageous developments are specified in the dependent claims and the description.

An aspect of the present disclosure relates to a container treatment system. The container treatment system has a container treatment device (e.g. container labeling device) with a (e.g. internal) treatment region for treating containers and a (e.g. external) protective enclosure which at least partially encloses the treatment region. The container treatment system has a, preferably optical and/or selectively switchable, sensor device which is configured to detect an intrusion (e.g. of an object such as a person) into a predefined, preferably selectively switchable, protective region in an environment of the container treatment device. The container treatment system has a processing device that is configured to adapt an operation of the container treatment device and/or the container treatment system depending on a signal output (e.g. regarding the detection of intrusion into the protective region) of the sensor device.

Advantageously, the protective region can be switched on or activated in certain situations and then act for example supplementary to, in addition to, or instead of the protective enclosure. For example, in the case of an automatic refitting of the container treatment device with an automatic format part change, in which the physical protective enclosure is opened and the container treatment device has to rotate, the (roomy) protective region can be activated in order to at least reduce the risk to operating personnel. Advantageously, the device can also make it possible to parallelize tasks performed automatically by robots, for example, and to be able to perform a plurality of tasks by the operator and the automatic system simultaneously without endangering the safety of the operator, since the operator can only be active outside the protective region, for example. This can advantageously enable faster refitting times and higher overall line efficiency.

In one embodiment, the protective enclosure has an openable portion, preferably a door, a window, a flap, or a lock, for making the treatment region accessible from outside the protective enclosure. Optionally, the sensor device can be configured such that the protective region is predefined such that it is adjacent to the openable portion. This can in particular advantageously reduce the potential risk for persons in the container treatment system resulting from the opened portion.

Preferably, the sensor device can be configured such that the protective region is predefined such that it at least partially encloses the container treatment device and/or the openable portion.

In an additional embodiment, the processing device is configured to switch on the sensor device and/or the protective region (e.g. to activate the sensor device and/or to activate processing of the signal output of the sensor device) when or before the openable portion is opened, preferably automatically. Advantageously, in this way the protective region can only be switched on if there is actually a potential danger to people in the system.

In an additional embodiment, the container treatment system further comprises an automatic changer which is designed for the automatic exchanging, maintenance, or refitting of (e.g. setup and/or format and/or fitting) parts in the treatment region of the container treatment device. Preferably, the automatic changer can be arranged outside the protective enclosure and can be designed to exchange, maintain, or change over the parts in the treatment region of the container treatment device through the openable portion. The switched-on protective region can thus advantageously prevent, for example, any danger to persons caused by the automatic changer and the container treatment device during refitting, etc.

In one embodiment, the processing device is configured to switch on the sensor device and/or the protective region (e.g. to activate the sensor device or to activate a processing of the signal output of the sensor device) when or before the automatic changer is activated for automatically exchanging, servicing, or changing over the parts in the treatment region of the container treatment device (e.g. manually or automatically). Advantageously, the protective region can therefore be particularly reliable in reducing or preventing any risk to persons posed by the automatic changer.

In an additional embodiment, the container treatment system further comprises a user interface (e.g. a free-standing touch column) which is arranged outside the predefined protective region and via which the sensor device and/or the protective region can be switched on. Preferably, the user interface can be a system user interface or a user interface of another container treatment device (e.g. filling device, closing device, or container manufacturing device) of the container treatment system. This can advantageously ensure that the protective region can only be switched on from outside the protective region. Advantageously, there is therefore no risk that a person who is already in the protective region and thus in the danger region will activate the protective region, since here-depending on the design of the sensor device-any intrusion into the protective region from outside would no longer be detectable.

In one variant embodiment, the container treatment system further comprises an additional user interface which is arranged within the predefined protective region and via which the sensor device and/or the protective region cannot be switched on, wherein the additional user interface is preferably a user interface of the container treatment device. This can advantageously further improve the protective design for preventing the protective region from being switched on from within the protective region.

In an additional variant embodiment, the sensor device has at least one camera and/or at least one signal scanner, preferably a laser scanner.

In one embodiment, the sensor device is configured to generate at least one (e.g. vertically aligned or horizontally aligned) signal curtain, preferably a light or laser curtain; and/or at least one signal barrier, preferably a light or laser barrier; and/or at least one (e.g. vertically or horizontally aligned) signal grid, preferably a light grid or laser grid, for detecting the intrusion. The signal curtain, the signal barrier and the signal grid can advantageously serve as outer boundaries of the protective region, so that intrusion into the protective region can be detected particularly reliably and quickly.

In an additional embodiment, the sensor device is configured to generate a plurality of signal curtains, a plurality of signal barriers, or a plurality of signal grids such that the plurality of signal curtains, the plurality of signal barriers, or the plurality of signal grids are arranged at an angle to one another and preferably adjoin one another or intersect one another. This advantageously enables an at least partial enclosing of the container treatment device by the protective region.

In one embodiment, the sensor device comprises an emitter, preferably a laser emitter, wherein the emitter is configured to output a preferably circular or fan-shaped (e.g. vertically or horizontally aligned) signal curtain, preferably a laser curtain. The sensor device further comprises a receiver, preferably a laser receiver, wherein the receiver is configured to detect when a signal beam, preferably a laser beam, of the signal curtain is reflected back from an object. In this way, a technology can advantageously be used in which the signal curtain can be positioned freely in the space as desired, since there are no restrictions due to the arrangement of the receiver.

Preferably, the receiver can be arranged directly next to the emitter.

It is also possible for the sensor device to have an additional emitter, preferably a laser emitter, wherein the additional emitter is configured to output an additional, preferably circular or fan-shaped (e.g. vertically or horizontally aligned) signal curtain, preferably a laser curtain. The sensor device can further comprise an additional receiver, preferably a laser receiver, wherein the additional receiver is configured to detect when a signal beam, preferably a laser beam, of the additional signal curtain is reflected back from an object.

Preferably, the signal curtain and the additional signal curtain can be oriented at an angle to each other and, for example, can adjoin each other or intersect.

In an additional embodiment, the processing device is configured to adapt the operation depending on the signal output such that an automatic refitting, part exchange, or maintenance operation of the container treatment device is stopped and/or a warning signal is issued when the signal output of the sensor device indicates intrusion into the predefined protective region, wherein preferably in the automatic refitting, part exchange, or maintenance operation, an openable portion of the protective enclosure is opened. The switched-on protective region can thus advantageously prevent danger to persons during refitting, part exchange, or maintenance operations.

In one variant embodiment, the processing device is configured such that the sensor device, the protective region, and/or a processing of the signal output of the sensor device is deactivated when the container treatment device is operated in an (e.g. normal) operating mode in which the protective enclosure and/or an openable portion of the protective enclosure is (e.g. completely) closed (unopened). Advantageously, a person can accordingly be permitted to stay near the container treatment device during normal operation to monitor the container treatment, since here the protective enclosure can safely protect the persons.

In an additional variant embodiment, the sensor device is configured to detect an intrusion and/or presence (e.g. of an object such as a person) in a predefined secure access corridor in the predefined protective region, or the sensor device is configured not to detect an intrusion and/or presence in a predefined secure access corridor in the predefined protective region as an intrusion into the predefined protective region. Advantageously, a corridor can thus be left out in the protective region so that the operator can be granted a certain access to necessary points near the container treatment device, for example to monitor the automatic refitting. Camera or scanner safety designs, for example, are particularly suitable for this purpose.

In an additional embodiment, the container treatment system further comprises at least one additional container treatment device which is arranged next to the container treatment device and is connected, for example, to the container treatment device for transporting containers. The sensor device can preferably be configured to predefine the protective region such that the at least one additional container treatment device delimits the protective region. Advantageously, this means that other machines in the system can also be used as physical outer boundaries of the protective region so that, for example, sensors can be spared.

Preferably, the container treatment system can be designed for controlling the temperature of, producing, cleaning, coating, testing, filling, closing, pasteurizing, labeling, printing, marking, laser marking, and/or packaging containers for liquid or pasty media, preferably beverages, liquid foodstuffs, or products of the pharmaceutical or healthcare industry.

For example, the containers can be realized as bottles, cans, canisters, cartons, vials, tubes, etc.

Preferably, the term “processing device” can refer to an electronic system (e.g. designed as a driver circuit or with microprocessor(s) and data memory) which, depending on the design, can perform control tasks and/or regulation tasks and/or processing tasks. Although the term “control” is used herein, this can also comprise or be understood as “regulate” or “feedback-control” and/or “process.”

The preferred embodiments and features of the invention described above can be combined with one another as desired.

### BRIEF DESCRIPTION OF THE DRAWINGS

Further details and advantages of the invention are described below with reference to the accompanying drawings. In the figures:

FIG. 1 is a perspective view of a schematically shown container treatment system according to one exemplary embodiment of the present disclosure; and

FIG. 2 is a plan view of the container treatment system of FIG. 1.

The embodiments shown in the drawings correspond at least in part, so that similar or identical parts are provided with the same reference signs and reference is also made to the description of other embodiments or figures for the explanation thereof to avoid repetition.

### DETAILED DESCRIPTION OF EMBODIMENTS

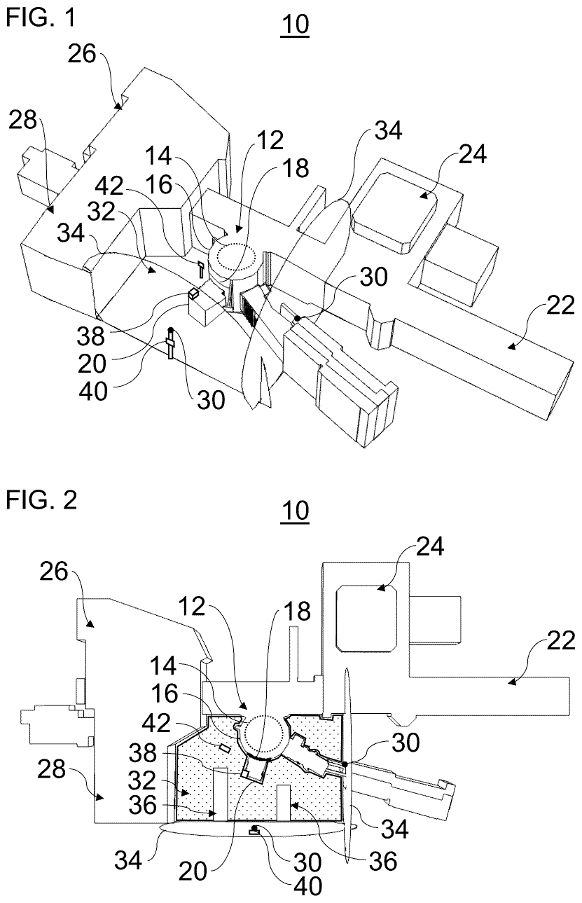

FIGS. 1 and 2 show a container treatment system 10.

The container treatment system 10 has a container treatment device 12, a sensor device 30, and a processing device 38. Optionally, the container treatment system 10 can, for example, also have an automatic changer 20, at least one further container treatment device 22, 24, 26, 28, and/or at least one user interface 40, 42.

The container treatment device 12 is designed to process containers. In the shown preferred embodiment, the container treatment device 12 is designed as a labeling device.

The labeling device can label containers. For example, the labeling device can comprise at least one labeling unit. The at least one labeling unit can be exchangeably capable of being coupled to and uncoupled from a periphery of the labeling device, e.g. via a preferably tool-free quick-coupling mechanism. The at least one labeling unit can be designed to apply labels, e.g. self-adhesive labels, cold glue labels, or roll labels.

It is possible for the container treatment device 12 to be designed for a different type of container treatment. For example, the container treatment device 12 can be designed to control the temperature of, manufacture, clean, coat, test, fill, close, pasteurize, print, mark, laser-mark, or package the containers. For example, the container treatment device 12 can be a preform heating oven, a container manufacturing device (e.g. container blowing machine), a coating device, a depalletizer, a rinsing device (e.g. with rinsing nozzles), a sterilizing device (e.g. with sterilization nozzles), a filling device (e.g. with filling valves), a closing device (e.g. with closing heads), a marking device (e.g. with laser markers), a printing device (e.g. with print heads), a pasteurization device, an adhesive container producing device (e.g. with adhesive application nozzles), a packaging device (e.g. with packing heads or film wrappers), and/or a palletizing device (e.g. with palletizing heads, sliding plates, or the like).

The container treatment device 12 can be rotatable by means of a drive. Preferably, the container treatment device 12 can be designed as a rotary container treatment device with a carousel that can be rotated by means of the drive. Containers can be held at a distance from one another on a circumference of the carousel in treating stations and treated (processed), e.g. labeled. Alternatively, the container treatment device 12 can be designed, for example, as a linear container treatment device, e.g. with only one treatment station or with a plurality of treatment stations arranged in series next to one another and/or one behind the other.

The container treatment device 12 can be connected to the at least one additional container treatment device 22, 24, 26, 28 by means of a container conveyor system. The container conveyor system can, for example, have at least one transport star and/or at least one linear conveyor.

The container treatment device 12 comprises a treatment region 14 and a mechanical protective enclosure 16.

In the treatment region 14, the containers are processed depending on the design of the container treatment device 12, e.g. labeled, etc.

The protective enclosure 16 partially, substantially completely, or completely encloses the treatment region 14. The treatment region 14 can be arranged protected inside the protective enclosure 16.

The protective enclosure 16 can, for example, have walls, windows, etc. to enclose the treatment region 14. The protective enclosure 16 can, for example, substantially have a cylinder-jacket-shaped or substantially a cylinder-jacket-segment-shaped outer wall, and optionally a roof.

Preferably, the protective enclosure 16 can have an openable portion 18. When the portion 18 is opened, it can provide access from outside the protective enclosure 16 to the treatment region 14. The portion 18 can for example be a door, a window, a flap, or a lock.

The protective enclosure 16 can be connected to an optional protective enclosure of the at least one further container treatment device 22, 24, 26, 28. For example, the protective enclosure 16 can merge into a protective enclosure of the container treatment device 24 and/or 26.

The automatic changer 20 is designed for the automatic exchanging, maintenance, or refitting of parts in the treatment region 14 of the container treatment device 12. The parts are preferably setup parts, such as container holders, container turntables, grippers, centering bells, etc., or any other parts, e.g. spare parts. During refitting, for example, setup parts of the container treatment device 12 can be exchanged in order to adapt the container treatment device 12 to a different container format.

Preferably, the automatic changer 20 has a handling device for carrying out work in the treatment region 14. Preferably, the handling device can have at least one tool, such as a gripper, for carrying out the work.

The handling device can be, for example, a stationary or mobile robot (e.g. exchanging robot) or any other type of handling device. Preferably, the handling device can have at least one pivotable and/or displaceable arm.

It is possible for the automatic changer 20 to have a magazine. Parts such as spare parts or setup parts can be accommodated in the magazine. Preferably, the handling device of the automatic changer 20 can move the parts from the magazine to the treatment region 14.

Preferably, the automatic changer 20 is arranged outside of the protective enclosure 16. The automatic changer 20 can change, service, or refit the parts in the treatment region 14 through the opened portion 18. For example, the handling device, e.g. its at least one arm and/or its at least one tool, can exchange, service, or refit the parts in the treatment region 14 through the opened portion 18.

The automatic changer 20 can preferably be operated in an automatic refitting, part exchange, or maintenance operation (or operating mode) of the container treatment device 12. In this automatic refitting, parts exchange, or maintenance operation, the portion 18 can be open. During refitting, parts exchange or maintenance operation, preferably no containers are processed in the treatment region 14. That is, the container treatment device 12 is not in normal operation/production operation.

As already mentioned, the container treatment system 18 can have at least one further container treatment device 22, 24, 26, 28. In the shown embodiment, the additional container treatment device 22 is, for example, a preform heating oven. The container treatment device 24 can be a container production device (e.g. container blow molding device). The container treatment device 26 can be a filling device for filling containers. The container treatment device 28 can be a closing device for closing containers.

The at least one additional container treatment device 22, 24, 26, 28 can be, for example, a rotary or a linear container treatment device.

The at least one additional container treatment device 22, 24, 26, 28 can be arranged next to the container treatment device 12.

The sensor device 30 is configured to detect an intrusion into a predefined protective region 32 in an environment of the container treatment device 12. For example, a person can walk into the specified protective region 32 and thus intrude into it. A signal output from the sensor device 30 to the processing device 38 can indicate that an intrusion into the predefined protective region 32 has been detected by the sensor device 30.

The protective region 32 can be predefined by an arrangement and orientation of the sensor device 30. The protective region 32 can, for example, be predefined such that it borders on the openable portion 18 and/or the protective enclosure 16. The protective region 32 can also be predefined, for example, such that it is delimited by the at least one further container treatment device 24, 26, 28.

The sensor device 30 is preferably an optical sensor device. Preferably, the sensor device 30 can, for example, have at least one camera and/or at least one scanner device, preferably a laser scanner.

The at least one camera can, for example, record the protective region 32. For example, image recognition algorithms can be applied to the camera recording. The image recognition algorithms can then be used to detect an intrusion of an object, such as a person, into the protective region 32.

The at least one laser scanner device can, for example, generate at least one laser curtain. The at least one laser curtain is preferably aligned vertically. The at least one laser curtain can externally delimit the protective region 32.

For example, the laser scanner device can include a laser emitter and a laser receiver. The laser emitter can be configured to emit a laser curtain, preferably circular or fan-shaped. The laser receiver can be configured to detect when a signal beam from the laser curtain is reflected back from an object. The laser receiver can preferably be arranged directly next to the laser emitter.

It is possible for the explained scanner design to be used using a technology other than laser technology in order to generally output a preferably circular or fan-shaped signal curtain by means of an emitter, and to detect by means of a receiver when a signal beam of the signal curtain is reflected back from an object. Here too, the receiver can preferably be arranged directly next to the emitter.

A scanning range of the scanner device, preferably a laser scanner device, can, for example, have a radius that lies in a range between 1000 mm and 15,000 mm, preferably between 2500 mm and 10,000 mm.

In general, the sensor device 30 can preferably be configured to generate at least one signal curtain 34 for detecting intrusion into the protective region 32. The signal curtain 34 can be, for example, a light curtain or a laser curtain. Alternatively or additionally, the sensor device 30 can, for example, be configured to generate at least one signal barrier, preferably a light or laser barrier, and/or at least one signal grid, preferably a light grid or laser grid, for detecting intrusion into the protective region 32.

The at least one signal curtain 34, the at least one signal barrier, and/or the at least one signal grid can externally delimit the protective region 32. An intrusion into the protective region 32 can be detected, for example, if the at least one signal curtain 34, the at least one signal barrier, and/or the at least one signal grid is disturbed or interrupted by an object.

Preferably, a plurality of signal curtains, a plurality of signal barriers and/or a plurality of signal grids are generated using a plurality of devices. These are preferably arranged at an angle to each other and adjoin one another or intersect one another, as shown by way of example in the figures.

For example, the sensor device 30 can be arranged at a height in a range between 2000 mm and 10,000 mm.

As shown by way of example in FIG. 2, at least one secure access corridor 36 can be arranged in the protective region 32. The at least one secure access corridor 36 can be predefined in such a way that a person is permitted to be present in this secure access corridor 36, for example to monitor the operation of the automatic changer 20, without there being a risk of injury to the person.

The sensor device 30 can be accordingly configured to detect an intrusion and/or presence in the at least one predefined secure access corridor 36 in the predefined protective region 32. For example, a signal output from the sensor device 30 to the processing device 38 can indicate that an intrusion and/or presence in the at least one predefined secure access corridor 36 has been detected by the sensor device 30.

The processing device 38 is configured to adapt an operation of the container treatment device 12 and/or the container treatment system 10 depending on a signal output from the sensor device 30 relating to the detection of the intrusion into the protective region.

For example, the processing device 38 can adapt the operation of the container treatment system 10 depending on the signal output such that an automatic refitting, part exchange, or maintenance operation is stopped by means of the automatic changer 20 when the signal output of the sensor device 30 indicates intrusion into the predefined protective region 32. When stopping, for example, the automatic changer 20 and/or the container treatment device 12 can be stopped. For example, a carousel of the container treatment device 12 that rotates slowly or in steps, compared to normal operation during refitting, part exchange, or maintenance operation, can be stopped.

Alternatively or additionally, an optical or acoustic warning signal can, for example, be issued.

If, however, the signal output from the sensor device 30 indicates only a detection of an intrusion and/or a presence in the secure access corridor 36 and not in the rest of the protective region 32, the processing device 38 can, for example, adapt the operation apart from this, e.g. by stopping the automatic refitting, exchange, or maintenance operation.

Alternatively, the sensor device 30 can, for example, be configured not to detect an intrusion and/or presence in a predefined secure access corridor 36 in the predefined protective region 32 as an intrusion into the predefined protective region 32 at all. Accordingly, a signal indicating detection of an intrusion into the protective region 32 would then not be output at all by the sensor device 30. Also accordingly, no adjustment of the operation is then made by means of the processing device 38.

Preferably, the sensor device 30 is only switched on depending on the situation.

For example, the processing device 38 can be configured such that the sensor device 30, the protective region 32, and/or a processing of the signal output of the sensor device 30 is deactivated when the container treatment device 12 is operated in an operating mode in which the openable portion 18 of the protective enclosure 16 is closed (unopened). This operating mode can, for example, be the normal operating mode of the container treatment device 12.

Preferably, the processing device 38 can be configured to switch on the sensor device 30 and/or the protective region 32 when or before the automatic changer 20 is activated for automatically changing, servicing, or refitting the parts in the treatment region 14 of the container treatment device 12. For example, a manual or automatic activation of the refitting, part exchange or maintenance operation can trigger the processing device 38 to switch on the sensor device 30.

The switching on of the sensor device 30 can, for example, comprise an activation of the sensor device 30. Alternatively or additionally, when switched on, processing of the signal output of the sensor device 30 can be activated by means of the processing device 38.

The user interface 40 is preferably arranged outside the predefined protective region 32. Preferably, the sensor device 30 and/or the protective region 32 can be switched on via a user input in the user interface 40, e.g. directly and/or by activating a refitting, part exchange, or maintenance operation of the container treatment device 12. The user interface 40 can preferably be a system user interface or a user interface of one of the additional container treatment devices 22, 24, 26 or 28.

On the other hand, the additional user interface 42 can be arranged within the predefined protective region 32. The sensor device 32 and/or the protective region 32 can preferably not be activatable via this further user interface 42, either directly nor indirectly. For example, the additional user interface 42 can be a user interface of the container treatment device 12, with which normal operation of the container treatment device 12 can otherwise be monitored and adjusted.

The at least one user interface 40, 42 can, for example, have a preferably touch-sensitive display, a loudspeaker, a signal light, a microphone, buttons and/or switches, etc.

The invention is not limited to the preferred exemplary embodiments described above. Rather, a plurality of variants and modifications are possible which likewise make use of the inventive concept and therefore fall within the scope of protection. In particular, the invention also claims protection for the subject matter and the features of the dependent claims, irrespective of the claims to which they refer. In particular, the individual features of independent claim 1 are each disclosed independently of one another. In addition, the features of the dependent claims are also disclosed independently of all of the features of independent claim 1 and, for example, independently of the features relating to the presence and/or configuration of the container treatment device, the treatment region, the protective enclosure, the sensor device, and/or the processing device of independent claim 1. All ranges specified herein are to be understood as disclosed in such a way that all values falling within the respective range are individually disclosed, e.g., also as the respective preferred narrower outer limits of the respective range.

### LIST OF REFERENCE SIGNS

10 container treatment system
12 container treatment device
14 treatment region
16 protective enclosure
18 openable portion
20 automatic changer
22 additional container treatment device
24 additional container treatment device
26 additional container treatment device
28 additional container treatment device
30 sensor device
32 protective region
34 signal curtain
36 access corridor
38 processing device
40 user interface
42 additional user interface

What is claimed is:

1. A container treatment system comprising:
a container treatment device with a treatment region for treating containers and a protective enclosure which at least partially encloses the treatment region;
a sensor device which is configured to detect an intrusion into a predefined protective region in an environment of the container treatment device; and
a processing device that is configured to adapt an operation of at least one of the container treatment device and the container treatment system depending on a signal output of the sensor device.

2. The container treatment system according to claim 1, wherein:
the sensor device is at least one of optical and selectively switchable;
the predefined protective region is selectively switchable;
the sensor device has a laser scanner; and
the sensor device is configured to generate any of at least one of a light and laser curtain, at least one of a light and laser barrier; and at least one of a light grid and laser grid for detecting the intrusion.

3. The container treatment system according to claim 1, wherein:
the protective enclosure has an openable portion for making the treatment region accessible from outside the protective enclosure.

4. The container treatment system according to claim 2, wherein:
the processing device is configured to switch on at least one of the sensor device and the protective region one of when and before the openable portion is opened.

5. The container treatment system according to claim 2, wherein one of:
the openable portion includes one of a door, a window, a flap, and a lock;
the sensor device is configured such that the protective region is predefined such that it adjoins the openable portion; and
the processing device is configured to switch on at least one of the sensor device and the protective region one of when and before the openable portion is automatically opened.

6. The container treatment system according to claim 1, also comprising:
an automatic changer that is designed for automatically one of changing, servicing, and refitting parts in the treatment region of the container treatment device,
wherein the automatic changer is arranged outside of the protective enclosure and is designed to one of exchange, maintain, and refit the parts in the treatment region of the container treatment device through the openable portion.

7. The container treatment system according to claim 6, wherein:
the processing device is configured to switch on at least one of the sensor device and the protective region one of when and before the automatic changer is activated for any of automatically exchanging, servicing, and refitting the parts in the treatment region of the container treatment device.

8. The container treatment system according to claim 6, further comprising:
an additional user interface which is arranged within the predefined protective region and via which at least one of the sensor device and the protective region cannot be switched on.

9. The container treatment system according to claim 1, also comprising:
a user interface which is arranged outside the predefined protective region and via which at least one of the sensor device and the protective region can be switched on.

10. The container treatment system according to claim 9, wherein at least one of:
the user interface is one of a system user interface and a user interface of an additional container treatment device of the container treatment system; and
the additional user interface is a user interface of the container treatment device.

11. The container treatment system according to claim 1, wherein:
the sensor device has any of at least one camera and at least one signal scanner.

12. The container treatment system according to claim 1, wherein:
the sensor device is configured to generate any of at least one signal curtain; at least one signal barrier; and at least one signal grid for detecting the intrusion.

13. The container treatment system according to claim 12, wherein:
the sensor device is configured to generate one of a plurality of signal curtains, a plurality of signal barriers, and a plurality of signal grids such that the one of the plurality of signal curtains, the plurality of signal barriers, and the plurality of signal grids are arranged at an angle to one another and one of adjoin one another and intersect one another.

14. The container treatment system according to claim 1, wherein the sensor device comprises:
an emitter being configured to output a signal curtain; and
a receiver configured to detect when a signal beam of the signal curtain is reflected back from an object.

15. The container treatment system according to claim 14, wherein at least one of:
the emitter includes a laser emitter;
the signal curtain includes one of a circular signal curtain, a fan-shaped curtain and a laser curtain;
the receiver includes a laser receiver; and
the signal beam includes a laser beam.

16. The container treatment system according to claim 1, wherein:
the processing device is configured to adapt the operation depending on the signal output such that one of an automatic refitting, part exchange, and maintenance operation of the container treatment device is stopped when the signal output of the sensor device indicates intrusion into the predefined protective region.

17. The container treatment system according to claim 16, wherein an openable portion of the protective enclosure is opened in one of the automatic refitting, the parts exchange, and the maintenance operation.

18. The container treatment system according to claim 1, wherein:
the processing device is configured such that at least one of the sensor device, the protective region and a processing of the signal output of the sensor device is deactivated when the container treatment device is operated in an operating mode in which at least one of the protective enclosure and an openable portion of the protective enclosure is closed.

19. The container treatment system according to claim 1, wherein one of:
the sensor device is configured to detect at least one of intrusion and presence in a predefined secure access corridor in the predefined protective region; and
the sensor device is configured not to detect at least one of an intrusion and presence in a predefined secure access corridor in the predefined protective region as an intrusion into the predefined protective region.

20. The container treatment system according to claim 1, also comprising:
at least one further container treatment device that is arranged next to the container treatment device,
wherein the sensor device is configured such that the protective region is predefined such that the at least one further container treatment device delimits the protective region.
